(19) United States
(12) Patent Application Publication
(10) Pub. No.: US 2007/0166405 A1
(43) Pub. Date: Jul. 19, 2007

(54) Extracts of houttuynia cordata and rubus coreanus and their composition for preventing and treating allergic diseases

(75) Inventors: Chang-Ho Song, Jeollabuk-do (KR); Ok-Hee Chai, Jeollabuk-do (KR); Yong-In Park, Busan (KR); Dong-Sin Lee, Gyeonggi-do (KR)

Correspondence Address: BUCHANAN, INGERSOLL & ROONEY PC, POST OFFICE BOX 1404, ALEXANDRIA, VA, 22313-1404, US

(73) Assignees: Chang-Ho Song, Jeonju (KR); REGEN BIOTECH INC., Seongnam (KR)

(21) Appl. No.: 10/587,677

(22) PCT Filed: Feb. 4, 2005
(86) PCT No.: PCT/KR05/00339

§ 371 Date: Jul. 27, 2006

(30) Foreign Application Priority Data
Feb. 5, 2004 (KR) 10-2004-0007404

(51) Int. Cl. A61K 36/28

(52) U.S. Cl. 424/725; 424/764; 424/774

(57) ABSTRACT

The present invention relates to an extract of Houttuynia cordata and Rubus coreanus, and a composition for the prevention and treatment of allergic diseases, comprising the same. The inventive extract and the composition comprising the same have the effect of preventing and treating allergic diseases by inhibiting the degranulation and histamine release of mast cells. Also, they show no cytotoxicity and thus can be safely use in vivo.

### TECHNICAL FIELD

The present invention relates to an extract of Houttuynia cordata and Rubus coreanus, and its composition and use for preventing and treating allergic diseases. More particularly, the present invention relates to water or organic solvent extract of houttuynia cordata and Rubus coreanus, a composition and a method for preventing and treating allergic diseases comprising the same.

### BACKGROUND ART

Recently, with an increase in environmental pollution and a change in residential environment, which are caused by industrial development, allergic diseases are increasing.

Allergy is the generic term for symptoms which are induced when the human immune system shows an extraordinarily sensitive reaction to foreign substances which show no reaction in most people. Clinical symptoms occurring during the allergic reaction process include specific immune reactions at the early stage, inflammatory reactions at the late stage, and the like. The specific immune reactions at the early stage are mostly mediated by mast cells and activated by a high-affinity immunoglobulin E (IgE) receptor (FcεRI) located on the cellular membrane of the mast cells.

The mast cells are widely distributed in the systemic organs, including the skin, the respiratory organ, the gastrointestinal mucosa, the brain, and around lymphatic vessels and blood vessels. When an IgE antibody bound to the IgE receptor located on the cellular membrane forms a bridge with an antigen or allergen introduced from the outside, the mast cells are then activated to degranulate, thus releasing chemical substances, such as histamine, heparin and protease, which are stored in granules within the mast cells. Among these chemical substances, histamine is most rapidly released so as to exhibit various actions, such as peripheral vasodilation, bronchial smooth muscle contraction, and the acceleration of gland cell secretion, thus causing immediate allergic reactions and inflammatory reactions.

Factors known to cause the degranulation of mast cells to be activated include: antigens; compound 48/80 (Panton, British Journal of Pharmacology, 1951); IgE antibodies; IgE receptor antibodies; IgE dimers; Con A; topical antibiotics polymyxin B; polylysine polypeptides; the stimulation of enzymes, such as alpha-chymotrypsin, and porcine pancreatic phospholipase A₂; the stimulation of Ca²⁺ coupled stimulation secretion (Ischizaka T et al., Proc. Natl. Acad. Sci. USA, 77, 1903, 1980); a change in cyclic nucleotide level; an increase in phosphorylation caused by the activation of protein kinase (Dagmar B et al., Cancer Research, 35, 2056, 1975); and the modification of the mast cell skeleton consisting of actin filaments, intermediate filaments and microtubules (Lagunoff D and Chi EY, J. Cell Biol. 71, 182, 1976).

Meanwhile, the chemical mediators released from the mast cells cause various allergic diseases, such as asthma, allergic rhinitis, allergic otitis, anaphylatic shock, and allergic skin disorder. Of them, the allergic skin disease is a common skin disorder and may hinder the emotional development, sound sleep and daily life of patients due to itching with chronic progression, thus causing severe mental and physical pains. The allergic skin diseases typically include atopic dermatitis, contact dermatitis, urticaria and psoriasis. Atopic dermatitis is not yet clearly known about its pathogenesis and is a chronic eczema occurring in 2-6 months old babies. It is most important for the treatment of atopic dermatitis to identify and to eliminate the allergy-causing substances from life. In the case of severe symptoms, either an antihistamine formulation as a systemic drug is administered or adrenal cortex hormone is locally applied. Contact dermatitis which is one kind of eczema is a hypersensitive reaction of the skin, which occurs when foreign substances are in contact with the skin. Contact dermatitis is divided according to the contact substances into sunlight contact dermatitis, mercury contact dermatitis and metal contact dermatitis. Urticaria is a phenomenon where edema occurs in the upper layer of the skin by an inflammation so that the skin temporarily swells up and a hypersensitive reaction of the skin accompanied by itching. Also, the urticaria is known to occur when various chemical mediators are released from mast cells and basophils by various causes and mechanisms, and act on the dermal microvasculature to dilate the microvasculature and to increase the vascular permeability so that a protein-rich liquid leaks out from the blood vessel to dermal tissue. Psoriasis is a chronic inflammatory skin disease characterized by the formation of various sizes of erythematous papules and plaques covered with silver-white scales, having clearly boundary, and by the epithelial proliferation in the histological view. Also, psoriasis is a disease of unknown cause where the improvement and deterioration of symptoms are repeated. Depending on the severity of psoriasis symptoms, various therapies have been developed and used. But it is difficult.

Currently, antihistamine agents or steroid agents are frequently used for the treatment of allergic diseases. However, these drugs mostly have a temporary effect and often have a severe side effect. Thus, there is a need for the development of a new therapeutic agent which has preventive and therapeutic effects on allergic diseases while having few side effects and sustained effects.

### DISCLOSURE OF THE INVENTION

Accordingly, during the development of a composition capable of effectively preventing or treating allergic diseases, the present inventors have selected substances having excellent anti-allergic activity from herbal extracts and found that the selected herbal extract have the effect of preventing or treating allergic diseases. On the basis of this finding, the present invention has been completed.

It is an object of the present invention is to provide a herbal extract having inhibitory activities against the degranulation and histamine release of mast cells by extracting Houttuynia cordata and Rubus coreanus with water or organic solvent.

Another object of the present invention is to provide the herbal extract for use as a medicament and a use of the herbal extract for preparing either a therapeutic agent against allergic diseases or an inhibitory agent against the degranulation and histamine release of mast cells.

Still another object of the present invention is to provide a pharmaceutical composition for the prevention or treatment of allergic diseases comprising the herbal extract as an active ingredient.

Still another object of the present invention is to provide a food composition for the prevention and improvement of allergic diseases comprising the herbal extract as an active ingredient.

Still another object of the present invention is to provide a method for the prevention or treatment of allergic diseases, which comprises the step of administering an effective amount of the herbal extract to a subject in need thereof.

Yet another object of the present invention is to provide a method for inhibiting the degranulation and histamine release of mast cell, which comprises the step of administering an effective amount of the herbal extract to a subject in need thereof.

To achieve the above objects, in one aspect, the present invention provides herbal extract having inhibitory activities against the degranulation and histamine release of mast cells, which is obtained by extracting Houttuynia cordata and Rubus coreanus with water or organic solvent.

In another aspect, the present invention provides the herbal extract for use as a medicament and a use of the herbal extract for preparing either a therapeutic agent against allergic diseases or an inhibitory agent against the degranulation and histamine release of mast cells.

In still another aspect, the present invention provides a pharmaceutical composition for the prevention or treatment of allergic diseases comprising the herbal extract as an active ingredient.

In still another aspect, the present invention provides a food composition for the prevention or improvement of allergic diseases comprising the herbal extract as an active ingredient.

In still another aspect, the present invention provides a method for the prevention or treatment of allergic diseases, which comprises the step of administering an effective amount of the herbal extract to a subject in need thereof.

In yet another aspect, the present invention provides a method for inhibiting the degranulation and histamine release of mast cells, which comprises the step of administering an effective amount of the herbal extract to a subject in need thereof.

Hereinafter, the present invention will be described in detail.

As used herein, the term “allergic diseases” means hypersensitivity reaction of the human body to any substance, i.e., diseases caused by hypersensitive reactions of the body's immune system to foreign substances. Preferably, this term means hypersensitivity reaction where mediator substances, such as histamine, are released by foreign substances so as to cause diseases. Examples of these allergic diseases include allergic asthma, allergic rhinitis, allergic otitis, anaphylactic shock and allergic skin disorders. The allergic skin disorders include atopic dermatitis, psoriasis, contact allergic dermatitis and urticaria.

In order to develop a composition capable of preventing or treating allergic diseases, the present inventors analyzed the antiallergic activities of 11 herbal extracts consisting of Mori folium extract, Arctii fructus extract, Schizandra chinensis extract, Lycium chinense extract, Cinnamomum cassia extract, Cornus officinalis Sieb. et Zucc extract, Crataegi fructus extract, Salicis Radicis Cortex extract, black sesame extract, Houttuynia cordata extract and Rubus coreanus extract. The antiallergic activities were determined by treating the peritoneal mast cells of rats with compound 48/80 known as a powerful substance for inducing the mast cell degranulation and then measuring whether the herbal extracts inhibit the mast cell degranulation induced by compound 48/80. From the test results, it could be seen that 5 herbal extracts consisting of Houttuynia cordata extract, Rubus coreanus extract, Cornus officinalis Sieb. et Zucc extract, Crataegi fructus extract, and Mori folium extract had the activity of inhibiting the compound 48/80-induced the mast cell degranulation of the rat peritoneal mast cells (see Test Example 1).

Moreover, the present inventors examined whether Houttuynia cordata extract, Rubus coreanus extract, Cornus officinalis Sieb. et Zucc extract, Crataegi fructus extract, and Mori folium extract confirmed to have the anti-allergic activity and cytotoxicity. The results could confirm that all the herbal extracts showed no cytotoxicity, indicating that they can be safely used in vivo (see Test Example 2).

Furthermore, the present inventors selected the Houttuynia cordata extract and Rubus coreanus extract having the highest anti-allergic activities among the five herbal extracts, and prepared a mixed extract of the selected Houttuynia cordata and Rubus coreanus, and analyzed the anti-allergic activity of the mixed extract.

In one test example of the present invention, the peritoneal mast cells of rats were pretreated with the mixed extract of Houttuynia cordata and Rubus coreanus and then compound 48/80. The results showed that the mixed extract had the effect of inhibiting the release of histamine from the mast cells. Also, it was shown that the histamine release-inhibitory effect of the mixed extract was remarkably excellent as compared to that of the Houttuynia cordata extract or the Rubus coreanus extract (see Test Example 3). From this effect, it is believed that the mixed extract increases the intracellular cyclic AMP level in the mast cells and reduces the uptake of calcium into cells, thus inhibiting the degranulation and histamine release of mast cells.

In another test example of the present invention, an anaphylactic shock mouse model was pretreated with the mixed extract of Houttuynia cordata and Rubus coreanus, and as a result, it could be seen that the mixed extract had a very excellent effect of inhibiting the death of the mice caused by anaphylactic shock and having 100% survival rate. Also, it was shown that the mixed extract of Houttuynia cordata and Rubus coreanus effectively inhibited the degranulation of the mesenteric mast cells of the anaphylactic shock mouse model (see Test Example 4).

In still another test example of the present invention, a rat model of cutaneous reaction was pretreated with the mixed extract of Houttuynia cordata and Rubus coreanus, and as a result, it could be seen that the mixed extract reduced a cutaneous reaction induced by compound 40/80 and effectively inhibited an increase in vascular permeability (see Test Example 5).

Furthermore, the present inventors examined the anti-allergic activity of a mixed extract comprising the Cornus officinalis Sieb. et Zucc, Crataegi fructus, and Mori folium confirmed to have anti-allergic activity in addition to the Houttuynia cordata and Rubus coreanus extracts. The results showed that the mixture had the activity of inhibiting compound 48/80-induced histamine release from the peritoneal mast cells of rats (see Test Example 6).

Furthermore, in still another test example of the present invention, either a capsule formulation comprising a mixed extract of Houttuynia cordata and Rubus coreanus or a capsule formulation comprising a mixed extract of Houttuynia cordata, Rubus coreanus, Cornus officinalis Sieb. et Zucc, Crataegi fructus and Mori folium was administered to a patient suffering from atopic dermatitis. Then, the concentrations of IgE and histamine in the patient's serum were measured and lesions were clinically observed. As a result, it could be seen that the inventive formulations had the same therapeutic effect as that of steroid agents and anti-histamine agents which have been used in the prior art (see Test Example 7).

Therefore, the mixed extract of Houttuynia cordata and Rubus coreanus according to the present invention is characterized in that it has the activity to effectively inhibit the degranulation and histamine release of mast cells, as well as the activities to inhibit the death caused by anaphylactic shock, and to decrease a cutaneous reaction induced by compound 40/80 and to effectively inhibit an increase in vascular permeability.

Thus, the inventive pharmaceutical composition for the prevention or treatment of allergic diseases is characterized by comprising the extracts of Houttuynia cordata and Rubus coreanus as active ingredients.

Furthermore, the inventive pharmaceutical composition for the prevention or treatment of allergic diseases is characterized by further comprising, in addition to the extract of Houttuynia cordata and Rubus coreanus, at least one selected from the group consisting of Cornus officinalis Sieb. et Zucc extract, Crataegi fructus exract, and Mori folium extract.

Preferably, the inventive pharmaceutical composition comprises an extract where Houttuynia cordata and Rubus coreanus are mixed at a weight ratio of 1:4 to 4:1. More preferably, the inventive pharmaceutical composition comprises an extract where Houttuynia cordata and Rubus coreanus are mixed at a weight ratio of 1:1. Also, if the inventive composition further comprises a Cornus officinalis Sieb. et Zucc extract, a Crataegi fructus extract, a Mori folium extract or a mixture thereof, this additional component may be contained at an amount of 1-20% by weight, and preferably 5-10% by weight, based on the total weight of the inventive composition.

Houttuynia cordata is a perennial herb of the genus Saururus chinensis and is widely distributed in the southeast region of Asia. Houttuynia cordata is known to have the effects of alleviating fever and treating swells and proctocele, and the activities to remove poison and to neutralize heavy metals.

Rubus coreanus Miq. is a deciduous broad-leaved shrub of the family Rosaceae, which is native to China and distributed in the Jejudo of Korea, the southern and middle regions of Korea, Japan, USA, Europe and the like. In Chinese medicine, the unripe fruit of Rubus coreanus is used. The known pharmacological effects of Rubus coreanus include: inhibition of reduction in eyesight by protecting the liver from being damaged by fatigue; promotion of urine excretion; improvement of ganacratia, insufficiency of semen, impotence and sexul function due to lack of stamina; heating of body; increasing stamina; promotion of hair growth; and preventing hair from turning gray.

Cornus officinalis Sieb. et Zucc is the fruit of Cornus officinalis tree, an deciduous tree of the family Cornaceae. It is effective against various adult diseases, such as kidney diseases, diabetes, hypertension, arthritis and women's diseases, and has nutritive, astringent, antibacterial and antifungal activities. It contains morroniside, loganin, sworoside, cornin, gallic acid, tartaric acid, malic acid and the like.

Crataegi fructus is the fruit of a plant of the family Rosaceae, and contains amygdalin, ursolic acid, chlorogenic acid, citric acid, racemic acid, flavonoid, vitamin C and the like. In the pharmacological effects of Crataegi fructus, it reduces the tones of the heart and blood vessels and has cardiac action. Also, it has the effects of the improvement of blood circulation, the promotion of digestion and the lowering of cholesterol content.

Mori folium is the leaf of a mulberry tree and known to have the effects of blood supplement and robustness (Shen Nong's Materia Medica). Also, it is known to be effective against diabetes, neuralgia and hypertension, and to have the effects of reducing stroke and lowering fever, and to be effective against tinnitus and headache (Chinese material medica). According to a recent study in Japan, it was found that the mulberry leaf has the effects of inhibiting an increase in blood glucose level and preventing diabetes.

Houttuynia cordata, Rubus coreanus, Cornus officinalis Sieb. et Zucc, Crataegi fructus and Mori folium used in the present invention may be collected from the nature or commercially obtained. The inventive composition may comprise a mixture obtained either by mixing the herbal materials and then extracting the mixed herbal materials together, or by extracting each of the herbal materials depending on the physical and chemical properties of the pharmacologically effective components thereof and then mixing the extracts with each other. The inventive herbal extracts may be prepared by a solvent extraction method known in the art. For example, extraction may be performed using one selected from the group consisting of water, alcohol such as ethanol and methanol, an organic solvent such as acetone, ethyl acetate, n-hexane, diethyl ether acetone or benzene, and a mixture thereof. If the extracts are prepared by the solvent extraction method, hot water extraction, ultrasonic extraction and reflux extraction methods may be used. In one example of the present invention, hot water extracts were prepared by adding purified water to the herbal materials and heating and extracting for a given time followed by filtration (see Example 1). In another example of the present invention, methanol extracts were prepared by adding 70% methanol to the herbal materials and extracting the resulting materials for a given time followed by filtration (see Example 2). The hot water extracts and the methanol extracts were examined for the inhibition of the compound 40/80-induced the mast cell degranulation of rats, and the results showed that they had no significant difference in the mast cell degranulation inhibition (see Test Example 1).

Meanwhile, the inventive pharmaceutical composition having the effect of preventing or treating allergic diseases may either comprise the herbal extracts alone or be formulated by adding at least one pharmaceutically acceptable carrier, excipient or diluent. As used herein, the term “pharmaceutically acceptable” means that the composition is physiologically acceptable, and when administered to the human body, it dose not cause allergic reactions, such as gastrointestinal disorders and dizziness, or similar reactions. Moreover, the inventive pharmaceutical composition formulated as described above may be administered for the prevention or treatment of allergic diseases via suitable routes. Suitable administration routes may include oral, intraocular, transdermal, subcutaneous, intravenous and intramuscular routes.

The inventive pharmaceutical composition may be formulated into an oral formulation or a parenteral formulation depending on a selected administration route. In the case of the parenteral formulation, the inventive pharmaceutical composition may be formulated into powders, granules, tablets, pills, sugar-coated tablets, capsules, liquids, gels, syrups, slurry, suspensions and the like, by a method known in the art. For example, the oral formulation may be obtained as tablets or sugar-coated tablets by blending the active components with a solid excipient, crushing the blend, adding suitable adjuvants, and then processing the mixture into a granular mixture. Examples of suitable excipients may include sugars, including lactose, dextrose, sucrose, sorbitol, mannitol, xylitol, erythritol and maltitol; starches, such as corn starch, wheat starch, rice starch and potato starches; celluloses, such as cellulose, methyl cellulose, sodium carboxymethylcellulose and hydroxypropylmethyl cellulose; and fillers, such as gelatin and polyvinylpyrrolidone. If necessary, a disintegrant, such as crosslinked polyvinylpyrrolidone, agar, alginic acid or sodium alginate, may be used. Furthermore, the inventive pharmaceutical composition may additionally comprise anticoagulants, lubricants, wetting agents, perfume, emulsifiers and/or preservatives. In the case of the parenteral formulation, the inventive pharmaceutical composition may be formulated in the form of injections, cream, lotion, external ointment, oil, moisturizers and nasal inhalers, by any method known in the art. For example, in the case of injections, the inventive composition may be formulated by dissolving the inventive herbal extracts in a physiologically acceptable buffer, such as Hanks solution, Ringer's solution or physiologically buffered saline.

As used herein, the term “effective amount” refers to the amount of a pharmaceutical composition which shows a preventive or therapeutic effect when administered to a patient. The effective amount of the pharmaceutical composition according to the present invention is preferably about 1-100 mg/kg body weight/day, and more preferably about 10-30 mg/kg body weight/day, based on the amount of the extract of Houttuynia cordata and Rubus coreanus. However, the dose of the inventive pharmaceutical composition may be suitably selected depending on various factors, such as administration routes, the age, sex, bodyweight and disease severity of patients.

Also, the inventive pharmaceutical composition may be administered one time or several times within the preferred range of its effective amount. However, the dose of the extract according to the present invention may be suitably selected depending on administration routes and subjects, the age, sex, bodyweight and disease conditions of patients. The composition containing the inventive extract is not specifically limited in its formulation and administration route and mode insofar as it shows the effects of the present invention.

As used herein, the term “subjects” means mammals, particularly animals including human beings. The subjects may also be patients requiring treatment.

The inventive pharmaceutical composition may be administered in combination with either a known compound having a preventive or therapeutic effect against allergic diseases, such as an immune regulator or an antihistamine agent, or an herbal extract, such as a mulberry root extract.

Moreover, the extract of Houttuynia cordata and Rubus coreanus may be provided in the form of a food composition for preventing or treating allergic diseases. Also, the food composition may further comprise at least one selected from the group consisting of Cornus officinalis Sieb. et Zucc extract, Crataegi fructus extract and Mori folium extract. In addition, the food composition may also contain a prior herbal extract known to prevent or improve allergic diseases, such as a mulberry root extract. The inventive food composition may include in all possible forms, such as functional food, nutritional supplement, health food and food additives. These forms of the food composition may be prepared by any conventional method known in the art.

For example, in case of the health food, the inventive herbal extracts themselves may be prepared into teas, juices or drinks for drinking, or granulated, capsulized or powdered for ingestion.

Also, the functional food may be prepared by adding the inventive herbal extracts to drinks (including alcoholic drinks), fruits and their processed foods (e.g., canned fruits, bottled fruits, jams, marmalades, etc), fishes, meats and their processed foods (e.g., ham, sausage, corned beef, etc.), breads and noodles (e.g., Japanese noodles, buckwheat noodles, ramycon, spaghetti, macaroni, etc.), fruit juices, various drinks, cookies, wheatgluten, milk products (e.g., butter and cheese), edible vegetable fat and oil, margarine, vegetable protein, retort foods, frozen foods, and various seasoning materials (e.g., soybean paste, soy, sauces, etc.).

Also, for use as the food additives, the inventive herbal extracts may be prepared into powder or a concentrate.

The content of the inventive herbal extracts in the inventive food composition is preferably 1-90% by weight, and more preferably 10-50% by weight, based on the total weight of the composition. Also, the inventive food composition comprises a mixed extract where Houttuynia cordata and Rubus coreanus are preferably mixed at a weight of 1:4 to 4:1.

Furthermore, the present invention provides the therapeutic use of extract of Houttuynia cordata and Rubus coreanus. Specifically, the present invention provides a method for preventing or treating allergic diseases, which comprises administering to an effective amount of an extract of Houttuynia cordata and Rubus coreanus to subjects in need thereof. Also, the present invention provides a method for inhibiting the degranulation and histamine release of mast cells, which comprises administering an effective amount of a extract of Houttuynia cordata and Rubus coreanus to subjects requiring in need thereof.

The present invention provides the herbal extract for use as a medicament, and a use of the herbal extract for preparation of either an agent for treating allergic diseases or an agent for inhibiting the degranulation and histamine release of mast cells.

The agent for treating allergic diseases or the agent for inhibiting the degranulation and histamine release of mast cells may further comprise a pharmaceutically acceptable carrier in addition to the extract of Houttuynia cordata and Rubus coreanus. Examples of the pharmaceutically acceptable carrier are as described above. Also, the inventive agent for treating allergic diseases may be administered orally or parenterally and examples of the orally or parenterally administration are as described above.

### BRIEF DESCRIPTION OF THE DRAWINGS

FIG. 1 shows optical microscopic photographs illustrating the results of observation for a change in the shape of rat peritoneal mast cells treated with the inventive herbal extract and compound 48/80, an histamine release inducer. (A: non-treated group; B: a group treated with compound 48/80; C: a group treated with a mixed extract of Houttuynia cordata and Rubus coreanus; and D: a group treated with a mixed extract of Houttuynia cordata and Rubus coreanus and compound 48/80.

FIG. 2 is graph showing the results of cytotoxicity tests for Houttuynia cordata extract, Rubus coreanus extract, Cornus officinalis Sieb. et Zucc extract, Crataegi fructus extract and Mori folium extract.

FIG. 3 is photographs showing lesions before and after treatment of allergic skin disease patients administered with a formulation comprising the inventive extract of Houttuynia cordata and Rubus coreanus extracts. (A: before treatment of an atopic dermatitis patient; B: after the treatment of the atopic dermatitis patient; C: before treatment of an atopic dermatitis patient; and D: after of the atopic dermatitis patient).

FIG. 4 is photographs showing lesions before and after treatment of an allergic skin disease patient administered with a prior therapeutic agent. (A: before treatment of an atopic dermatitis patient; and B: after treatment of the atopic dermatitis patient).

### BEST MODE FOR CARRYING OUT THE INVENTION

Hereinafter, the present invention will be described in detail by the following examples. It is to be understood, however, that these examples illustrate the present invention and are not construed to limit the scope of the present invention. In the following examples, percentages for a solid/solid mixture, a liquid/liquid mixture and a liquid/solid mixture are based on weight/weight, volume/volume and weight/volume, respectively, and unless otherwise stated, all reactions were performed at room temperature.

### EXAMPLE 1

### Preparation of Hot Water Extracts of Herbal Materials

In order to select herbal extracts having antiallergic activity, each of hot water extracts of Mori folium, Arctii Fructus, Schizandra chinensis, Lycium chinense, Cinnamomum cassia, Cornus officinalis Sieb. et Zucc, Crataegi fructus, Salicis Radicis Cortex, black sesame, Houttuynia cordata and Rubus coreanus was prepared. The herbal materials used in the above preparation were all purchased from Korean Agricultural Cooperatives in a dried state.

First, the herbal materials were washed clean and cut into a consistent size of less than 1×1×1 cm. 300 g of each of the herbal materials was added to 3 liters of purified water and extracted under pressure at 100° C. for 4 hours. After completion of the extraction, the each of the extracts was filtered through a 100-mesh filter membrane, and the filtrate was concentrated to 25 brix with a rotary vacuum evaporator (EYELA, Tokyo, Rikakikai Co. Ltd.). The concentrate was dried under vacuum and powdered to a size of 80 meshes. The dried powder was diluted in saline at various concentrations and used in tests.

### EXAMPLE 2

### Preparation of Methanol Extracts of Herbal Materials

Each of Mori folium, Arctii Fructus, Schizandra chinensis, Lycium chinense, Cinnamomum cassia, Cornus officinalis Sieb. et Zucc, Crataegi fructus, Salicis Radicis Cortex, black sesame, Houttuynia cordata and Rubus coreanus was washed clean and cut into a constant size of less than 1×1×1 cm. 500 g of each of the herbal materials was added to 5 liters of 70% methanol and extracted at 50° C. for 72 hours. After completion of the extraction, filtration and vacuum drying were conducted in the same manner as in Example 1 to yield methanol extracts.

### TEST EXAMPLE 1

### Selection of Herbal Extracts Having Antiallergic Activity

Each of the hot water extracts and methanol extracts of Mori folium, Arctii Fructus, Schizandra chinensis, Lycium chinense, Cinnamomum cassia, Cornus officinalis Sieb. et Zucc, Crataegi fructus, Salicis Radicis Cortex, black sesame, Houttuynia cordata and Rubus coreanus was examined for antiallergic activity.

For this purpose, mast cells were harvested from the abdominal cavity of rats. The mast cells were treated with the herbal extracts and compound 48/80, a histamine release inducer. Then, the shape of the mast cells was observed and the amount of histamine release from the mast cells was measured.

1-1) Harvest of Mast Cells from Abdominal Cavity of Rats

Mast cells were harvested by the following method modified and supplemented from a method known in the art (Cochrane D E and Douglas W W, Proc. Natl. Acad Sci. USA, 71, 408, 1974). Healthy and mature Sprague-Dawley male rats weighing about 250-300 g were anesthetized with ether and killed by receiving a hard blow on the back of the head. Then, about 10 ml of HEPES-Tyrode buffer was injected into the abdominal cavity of the rats, and the abdominal wall was lightly massaged for 90 seconds. The central line of the abdominal wall was incised and the peritoneal lavage fluid was taken with a spoid and centrifuged at 200×g for 10 minutes. The supernatant is discarded and the remaining substance was re-suspended in a HEPES-Tyrode buffer to 1×10⁶ cells/ml. This mast cell suspension was used for observation of the mast cells. Pure isolation of the mast cells from the peritoneal mast cell suspension was performed by a known method (Hachisuka et al., Arch. Dermatol. Res., 280:358, 1988). 3.5 ml of an isotonic percoll solution (1 ml of 10× Hank's solution and 9 ml of percoll) was added to a 15-ml centrifugal test tube. 0.75 ml of the mast cell suspension was carefully put on the isotonic percoll solution, and 0.5 ml of HEPES-Tyrode buffer was filled in the upper layer of the tube. Then, the contents within the test tube were left to stand for about 10 minutes followed by centrifugation at 125×g for 15 minutes. After the centrifugation, 2 ml of the supernatant was removed by a pipette and washed two times with HEPES-Tyrode buffer of 4° C., thus prepare a pure mast cell suspension. The viability and purity of the mast cells were measured using the Trypan blue and Kimura stain solutions and only the suspensions with a mast cell viability and purity of more than 90% were used in tests.

1-2) Observation of Shape of Rat Peritoneal Mast Cells Caused by Treatment with Herbal Extracts

Twenty five microliter of a hot water extract or methanol extract of each of Mori folium, Arctii Fructus, Schizandra chinensis, Lycium chinense, Cinnamomum cassia, Cornus officinalis Sieb. et Zucc, Crataegifructus, Salicis Radicis Cortex, black sesame, Houttuynia cordata and Rubus coreanus was treated with 200 μl of the abdominal mast cell suspension obtained in Test Example 1-1) together with 25 μl of HEPES-Tyrode buffer, and allowed to react in an incubator at 37° C. for 30 minutes. Also, in order to determined whether the herbal extracts have the activity to inhibit the mast cell degranulation induced by compound 48/80, 25 μl of the hot water extract or methanol extract of each of the herbal extracts was added to the mast cell suspension and allowed to react in an incubator at 37° C. for 10 minutes. And then the reaction substance was added with 25 μl of compound 48/80 and allowed to react in an incubator at 37° C. for 20 minutes.

For use as a positive control group for comparison with the test group, 200 μl of the mast cell suspension was treated with 25 μl of HEPES-Tyrode buffer and 25 μl (5 μl/ml) of compound 48/80 and allowed to react in an incubator at 37° C. for 30 minutes. As a negative control group, the mast cell suspension with no treatment was used.

After completion of the reaction, 200 μl of the mast cell culture was dropped onto a slide glass (22×60 mm) placed on the stage of an inverted microscopy, and then left to stand at room temperature for 10 minutes in order to the mast cells could be precipitated. Next, the mast cells were observed under a magnification of ×1,000.

Generally, normal mast cells are mostly circular or oval in shape, clearly outlined and have many granules filled in the cytoplasm. The diameter of the mast cells is about 10-20 μm which is more than two times larger than that of other cells (lymphocytes or neutrophil leukocytes). Thus, mast cells which are circular or oval in shape, clearly outlined and have granules of high optical refractive index filled in the cytoplasm, was classified as normal mast cells. On the other hand, mast cells where the cellular outline is unclear and granules in the cytoplasm are either protruded from the cell surface or scattered around the cells was classified as degranulated mast cells.

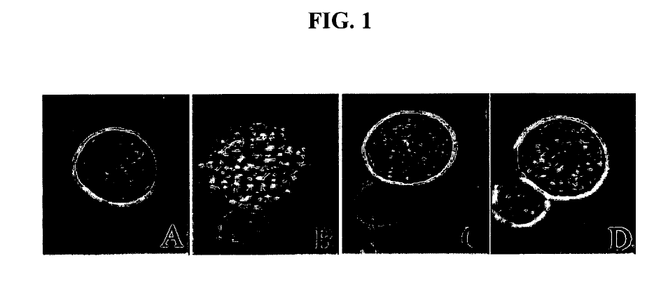

In the test results, it could be seen that, in the case of the negative control group with no treatment, the size of mast cells was about two times larger than that of lymphocytes in the suspension, and the mast cells were circular or oval in shape. Moreover, findings in the normal mast cells were shown clearly outlined, and the cytoplasm was filled with granules having high optical refractive index so that the nuclei were not clearly observed (see A of FIG. 1).

In the case of the positive control group treated with the compound 48/80 solution, the degranulation phenomena could be observed in which the optical refractive index of granules in the cytoplasm was reduced within a few minutes, and the cells were gradually swollen so that the membrane of cell became irregular and at the same time, the granules with reduced refractive index were protruded from the cell surface (see B of FIG. 1).

In the case of the mast cell treated with only the hot water extract of each herbal materials, the shape, size and surface outline of the mast cells were not significantly different from the findings in the normal mast cells, and during 30 minutes of the addition of the hot water extracts, other changes could not be observed (C of FIG. 1).

Meanwhile, in the case where the mast cell was treated with a hot water extract of each of Houttuynia cordata, Rubus coreanus, Cornus officinalis Sieb. et Zucc Crataegi fructus and Mori folium and then added with compound 48/80 solution, it was shown that the mast cell degranulation was inhibited so that their shape, size and surface outline were the same as in the normal mast cells (see D of FIG. 1). In addition, in the case of treatment with a methanol extract of each of Houttuynia cordata, Rubus coreanus, Cornus officinalis Sieb. et Zucc, Crataegi fructus and Mori folium, it could also be seen that the mast cell degranulation was inhibited (data not shown). On the other hand, when the mast cell culture was treated with a hot water extract or methanol extract of each of Arctii Fructus, Schizandra chinensis, Lycium chinense, Cinnamomum cassia, Salicis Radicis Cortex and black sesame and then added with compound 48/80 solution, the mast cell degranulation was not inhibited (see Table 1).

From the above test results, it can be seen that the hot water extract or methanol extract of each of Houttuynia cordata, Rubus coreanus, Cornus officinalis Sieb. et Zucc, Crataegi fructus and Mori folium does not influence the shape of the mast cells, but inhibits the compound 48/80-induced the mast cell degranulation and allows the shape of the mast cells to be maintained at the shape of the normal mast cells.

TABLE 1
Inhibition of compound 48/80-induced the mast cell degranulation by
treatment with hot water extracts or methanol extracts of herbal extracts

| | Mast cell degranulation | |
|---|---|---|
| Kind of herbal materials | Hot water extracts | Methanol extracts |
| Mori folium | − | − |
| Arctii fructus | + | + |
| Schizandra chinensis | + | + |
| Lycium chinense | + | + |
| Cinnamomum cassia | + | + |
| Cornus officinalis Sieb. et Zucc | − | − |
| Crataegi fructus | − | − |
| Salicis Radicis Cortex | + | + |
| black sesame | + | + |
| Houttuynia cordata | − | − |
| Rubus coreanus | − | − |

+ the mast cell degranulation was observed.
− the mast cell degranulation was not observed.

### TEST EXAMPLE 2

### Cytotoxicites of Houttuynia cordata Extract, Rubus coreanus Extract, Cornus officinalis Sieb. et Zucc Extract, Crataegi fructus Extract and Mori folium Extract

The cytitoxicities of Houttuynia cordata extract, Rubus coreanus extract, Cornus officinalis Sieb. et Zucc extract, Crataegi fructus extract and Mori folium extract which have been conformed to anti-allergic activity in Test Example 1-2) were examined.

To 225 μl (7×10⁵ cells/0.225 ml) of the white mouse abdominal suspension containing mast cells and other cells obtained in Example 1-1), 25 μl of 100 mg/ml of each of Houttuynia cordata extract, Rubus coreanus extract, Cornus officinalis Sieb. et Zucc extract, Crataegi fructus extract and Mori folium extract was added. Then, the mast cell suspension was allowed to react at 37° C. for 2 hours and examined for cell viability. For use as a control group, the mast cell suspension was treated with buffer in place of the extract. The reaction solution was centrifuged 400×g at 4° C., and the supernatant was discarded. The remaining cells were added with 50 μl of MTT (1 mg/ml of 3-(4,5-dimethyl thiaazol-2-yl)-2,5-diphenyl-tetrazolium bromide, Sigma-Aldrich) and allowed to react at 37° C. for 1 hour. After completion of the reaction, 100 μl of DMSO (dimethyl sulfoxide, C₂H₆SO, Sigma-Aldrich) was added. Then, the mixture was measured for optical density (O.D.) at 570 nm with a spectrophotometer, and, the cell viability was calculated as follows:
Viability (%)=(optical density of test group/optical density of control group)×100

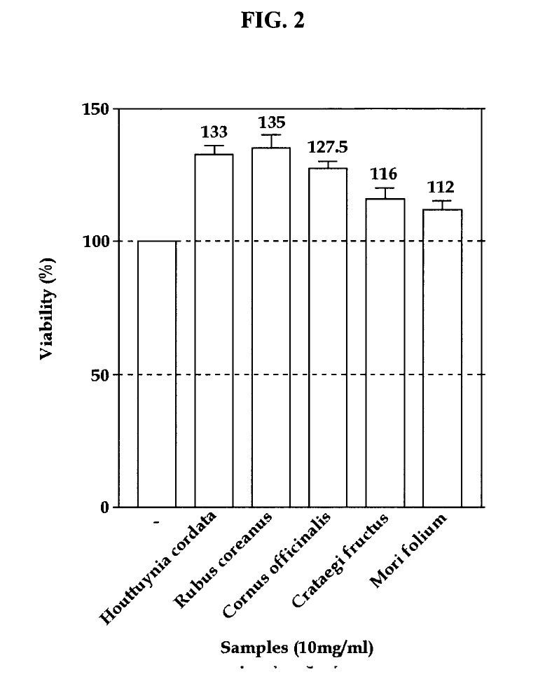

In the test results, the Houttuynia cordata extract, Rubus coreanus extract, Cornus officinalis Sieb. et Zucc extract, Crataegi fructus extract and Mori folium extract showed no cytotoxicity (see FIG. 2).

### EXAMPLE 3

### Preparation of Mixed Extract of Houttuynia cordata and Rubus coreanus

Houttuynia cordata and Rubus coreanus were mixed with each other at a weight ratio of 1:1, from which a mixed extract was prepared in the following manner. Dried Houttuynia cordata and Rubus coreanus were washed clean and cut into a constant size of less than 1×1×1 cm. Then, the cut herbal materials were mixed at an amount of 150 g for each material. 300 g of the mixed herbal materials were put in 3 liters of purified water and extracted under pressure at 100° C. for 4 hours.

### TEST EXAMPLE 3

### Examination of Effect of Mixed Extract of Houttuynia cordata and Rubus coreanus on Inhibition of Histamine Release from Rat Peritoneal Mast Cells

In order to examine if the mixed extract of Houttuynia cordata and Rubus coreanus prepared in Example 3 has the effect of inhibiting histamine released from abdominal mast cells by compound48/80, the following tests were performed. The histamine release inhibitory effect was measured on the rat peritoneal mast cells harvested in Test Example 1-1). First, to measure the amount of histamine released from normal mast cells, 200 μl of the peritoneal mast cells were treated with 50 μl of saline. Also, to measure the amount of histamine released by compound 48/80, the peritoneal mast cells were added with 25 μl of saline, and after 10 minutes, added with 25 μl of compound 48/80 solution (5 μg/ml). In order to examine if the Houttuynia cordata extract, the Rubus coreanus extract and the mixed extract of Houttuynia cordata and Rubus coreanus induce histamine to be released from mast cells, the mast cells were added with 25 μl of 1 mg/ml of each extract, and after 10 minutes, added with 25 μl of saline (final concentration of 0.1 mg/ml). Moreover, in order to examine if the herbal extracts inhibit the release of histamine from mast cells induced by compound 48/80, the mast cells were treated with each of the extract in the same manner as described above, and after 10 minutes, added with 25 μl of compound 48/80 solution (5 μg/ml).

After completion of the reaction, the reaction solution was centrifuged at 4° C. and 400×g, and the supernatant was collected and measured for the amount of histamine released from mast cells by a method modified from a known method (Harvima et al., Clinica Chimica Acta. 171:247, 1988). In other words, 10 μl of the collected supernatant, 1.5 μl of S-adenosyl (methyl-¹⁴C) methionine (2 μCi/ml), 40 μl of 300 mM Tris-glycin buffer (pH 8.3), and 5 μl of histamine N-methyl transferase, were reacted with each other in an incubator at 37° C. for 90 minutes, and the reaction was stopped by the addition of 20 μl of 3N perchloric acid. To neutralize the perchloric acid, 20 μl of 10N NaOH was added, and 1 ml of tolune-isoamyl alcohol was added, and the mixture was extracted to obtain 700 μl of a supernatant. To the supernatant, 3 ml of Cocktail solution (ULTIMA GOLD™, Packard Bioscience Company, USA) was added. Next, counter per minute (CPM) was measured using a β-counter (Liquid scintillation Analyzer, Acanberra company, Australia), and the amount of histamine was measured by a histamine standard curve. The amount of histamine was expressed as a percentage based on the total amount of histamine. The total amount of histamine was determined by treating 200 μl of the abdominal mast cells with 50 μl of saline to obtain 250 μl of a mast cell suspension, heating the mast cell suspension at 100° C. for 10 minutes, centrifuging the heated suspension, and determining the amount of histamine measured from the supernatant as 100. Histamine release (%) was calculated by the following equation:
Histamine release (%)=(amount of histamine release in test group/total amount of histamine release)×100

From the amount of histamine calculated as described above, the inhibition of histamine release from mast cells by treatment with the herbal extracts was calculated by the following equation:
Inhibition of histamine release (%)=[1-(amount of histamine release by treatment with herbal extract and compound 48/80−amount of histamine release by treatment with herbal extract)/(amount of histamine release by treatment with compound 48/80−amount of histamine release by treatment with saline)]×100

In the test results, in the case of the mast cell suspension treated with only saline, the histamine release was 2.7%. In the case of the mast cells treated with the Houttuynia cordata extract, the Rubus coreanus extract or the mixed extract of Houttuynia cordata and Rubus coreanus, the histamine release was similar to that in the case treated with only saline. On the other hand, in the case of the mast cells treated with only compound 48/80, the histamine release was as high as 60.2%. Also, in the case of the mast cells treated with the hot water extract of the herbal material and then with compound 48/80, it could be found that the histamine release from the mast cells induced by compound 48/80 was inhibited.

In other words, in the case of the mast cells treated with the mixed extract of Houttuynia cordata and Rubus coreanus and then with compound 48/80, the histamine release was 9.4±1.6%, and the inhibition of histamine release was 88%. On the other hand, in the case of the mast cells treated with the Houttuynia cordata extract alone and then with compound 48/80, the histamine release was 41.0±2.1%, and the inhibition of histamine release was 33%. In the case of the mast cells treated with the Rubus coreanus extract alone and then with compound 48/80, the histamine release was 37.4±3.2%, and the inhibition of histamine release was 39% (see Table 2).

From the above test results, it can be seen that the mixed extract of Houttuynia cordata and Rubus coreanus is excellent in the effect of inhibiting the compound 48/80-induced histamine release of the mast cells, as compared to the case of treatment with the Houttuynia cordata extract alone or the Rubus coreanus extract alone.

TABLE 2
Inhibition of compound 48/80-induced histamine release from mast cells by
treatment with mixed extract of Houttuynia cordata and Rubus coreanus

| | | | Inhibition of |
|---|---|---|---|
| Kind of extract | Compound 48/80 | Histamine release (%) | histamine release (%) |
| Saline | − | 2.7 ± 0.3¹⁾ | |
| | + | 60.2 ± 3.2 | |
| Houttuynia cordata | − | 2.5 ± 0.2 | |
| | + | 41.0 ± 2.1 | 33 |
| Rubus coreanus | − | 2.4 ± 0.4 | |
| | + | 37.4 ± 3.2 | 39 |
| Mixed extract of | − | 3.9 ± 0.3 | |
| Houttuynia cordata and | + | 9.4 ± 1.6 | 88 |
| Rubus coreanus | | | |

+ treated,
− untreated
¹⁾each value is expressed as mean ± standard error (n = 10)

### TEST EXAMPLE 4

### Examination of Antiallergic Activity of Mixed Extract of Houttuynia cordata and Rubus coreanus Using Anaphylactic Shock Mouse Model

The antiallergic activity of the mixed extract of Houttuynia cordata and Rubus coreanus was examined using an anaphylactic shock mouse model. The anaphylactic shock mouse model was obtained by administering compound 48/80 into the abdominal cavity of ICR mice to induce anaphylactic shock. In this test, the effects of the treatment of the anaphylactic shock model with the inventive mixed extract of Houttuynia cordata and Rubus coreanus on the mortality of mice and the degranulation of mesenteric mast cells of mice were examined.

4-1) Examination of Effect of Mixed Extract of Houttuynia cordata and Rubus coreanus on Mortality of Mice Caused by Anaphylactic Shock

Anaphylactic shock in mice was caused by a known method using compound 48/80 (Byoung-Duek, Jeon et al., The Korean Journal of Biological Response Modifier, 2, 169, 1992). Healthy ICR mice weighing about 20-30 g were selected and compound 48/80 was administered into the abdominal cavity of the mice one time at an amount of 15 μg/g bodyweight. As a control group, saline in place of compound 48/80 was injected. To the mice, 300 μl of 10 mg/ml or 1 mg/ml of each of the Houttuynia cordata extract, the Rubus coreanus extract and the mixed extract of Houttuynia cordata and Rubus coreanus was administered three times into the abdominal cavity compound at 24 hours, 12 hours and 1 hour before administration of compound 48/80. Also, the mice were administered with only compound 48/80 without pretreatment with the extract and then observed for at least 3 days until the mice were dead. The mortalities of mice by the Houttuynia cordata extract, the Rubus coreanus extract, the mixed extract of Houttuynia cordata and Rubus coreanus, and compound 48/80, were calculated by dividing the number of mice dead due to anaphylactic shock by the total number of mice used in the tests. Moreover, the average time (minutes) taken for mice to die due to anaphylactic shock after injection with compound 48/80 was measured.

In the test results, the mice administered with only saline without administration with compound 48/80 all survived. Also, the mice administered with saline along with compound 48/80 were all dead (100% mortality), and the average time taken to die was 17.8 minutes.

In the case of the mice administered with the Houttuynia cordata extract before administration with compound 48/80, the mortality was 30% (Houttuynia cordata extract concentration of 10 mg/ml) or 70% (Houttuynia cordata extract concentration of 1 mg/ml), and the average time taken to die was 57.4 minutes (Houttuynia cordata extract concentration of 10 mg/ml) or 42.3 minutes (Houttuynia cordata extract concentration of 1 mg/ml).

Also, in the case of the mice administered with the Rubus coreanus extract before administration with compound 48/80, the mouse mortality was 30% (Rubus coreanus extract concentration of 10 mg/ml) or 80% (Rubus coreanus extract concentration of 1 mg/ml), and the average time taken to die was 53.4 minutes (Rubus coreanus extract concentration of 10 mg/ml) or 39.8 minutes (Rubus coreanus extract concentration of 1 mg/ml).

Meanwhile, in the case of the mice administered with the mixed extract of Houttuynia cordata and Rubus coreanus before administration with compound 48/80, the mouse mortality 0% (all survived; concentration of mixed extract of 10 mg/ml) or 10% (concentration of mixed extract of 1 mg/ml), and the time taken to die was 65.5 minutes (see Table 3).

In the above test results, it can be seen that, even when the Houttuynia cordata extract or the Rubus coreanus extract is used alone, the mouse death caused by anaphylactic shock induced by compound 48/80 can be inhibited, but when the mixed extract of Houttuynia cordata and Rubus coreanus is used, the mouse death can be effectively inhibited. Also, the higher the treatment concentration of the extract, the inhibitory effect against the mouse death caused by anaphylactic shock induced by compound 48/80 is more excellent.

TABLE 3
Inhibition of anaphylactic shock-caused mouse mortality by mixed extract of
Houttuynia cordata and Rubus coreanus

| | | | Number of dead | | Average time |
|---|---|---|---|---|---|
| | | Compound | mice/number of | Mortality | taken to die |
| Treatment (mg/ml) | | 48/80 | mice used in test | (%) | (minute) |
| Saline | | − | 0/10 | 0 | — |
| Saline | | + | 10/10 | 100 | 17.8 |
| Houttuynia cordata | 10 | + | 3/10 | 30 | 57.4 |
| extract | 1 | + | 7/10 | 70 | 42.3 |
| Rubus coreanus | 10 | + | 3/10 | 30 | 53.4 |
| extract | 1 | + | 8/10 | 80 | 39.8 |
| Mixed extract of | 10 | + | 0/10 | 0 | — |
| Houttuynia cordata | 1 | + | 1/10 | 10 | 65.5 |
| and Rubus coreanus | | | | | |

+ treated,
− untreated

4-2) Examination of Effect of Mixed Extract of Houttuynia cordata and Rubus coreanus on Anaphylactic Shock-caused Degranulation of Mesenteric Mast Cells of Mice

According to the same method as described in Example 4-1), only compound 48/80 was injected into the abdominal cavity of mice, or mice were pretreated three times with each of the Houttuynia cordata extract, the Rubus coreanus extract and the mixed extract of Houttuynia cordata and Rubus coreanus and then administered with compound 48/80. After 15 minutes, the mice were sacrificed by cervical dislocation.

The central line of the abdominal wall of the sacrificed mice was incised, and methanol was injected directly into the abdominal cavity followed by fixing for 20 minutes. The fixed mesentery was taken and put on a slide. Then, it was washed with water, stained with 0.1% toluidine blue (pH 4.0) for 1 minute, washed with water, dewatered and sealed. For each animal, two slides were prepared. The degranulation phenomena of the mast cells were observed under a ×400 optical microscope, and the mast cells were classified into normal mast cells, moderately degranulated mast cells and severely degranulated mast cells. The number of the mast cells was counted to calculate the degranulation index of the mast cells. In this case, nondegranulated mast cells were classified as normal mast cells, mast cells around which some granules have existed were classified as moderate degranulation, and mast cells around which many granules have been scattered were as severe degranulation. The number of the mast cells per segment was randomly counted two times over 10 fields for each time and averaged. In order to reduce individual errors, findings observed by two persons for the same sample were integrated and averaged. Degranulation index (%) and inhibition of degranulation (%) were calculated by the following equations:
Degranulation index (%)=[(number of normal mast cells×0)+(number of moderately degranulated mast cells×50)+(number of severely degranulated mast cells×100)]/total number of mast cells
Inhibition of degranulation (%)=[1-(degranulation index by mixed extract of Houttuynia cordata and Rubus coreanus and compound 48/80/degranulation index by compound 48/80)]×100

In the test results, the mast cell degranulation index in the control group administered with only saline was 6.3, and the mast cell degranulation index in the mouse group administered with only compound 48/80 was 92.7.

In the case of the mice administered with the Houttuynia cordata extract before administration with compound 48/80, the mast cell degranulation index was 42.0 (Houttuynia cordata extract concentration of 10 mg/ml) or 82.1 (Houttuynia cordata extract concentration of 1 mg/ml). Also in this case, the inhibition of mast cell degranulation was 54.6% (Houttuynia cordata extraction concentration of 10 mg/ml) or 11.4% (Houttuynia cordata extraction concentration of 1 mg/ml).

In the case of the mouse group administered with the Rubus coreanus extract before administration with compound 48/80, the mast cell degranulation index was 42.7 (Rubus coreanus extract concentration of 10 mg/ml) or 80.6 (Rubus coreanus extract concentration of 10 mg/ml), and the inhibition of mast cell degranulation was 53.9% (concentration of 1 mg/ml) or 13% (concentration of 1 mg/ml).

Also in the case of the mouse group administered with the mixed extract of Houttuynia cordata and Rubus coreanus before administration with compound 48/80, the mast cell degranulation index was 9.1 (concentration of mixed extract of 10 mg/ml) or 27.2 (concentration of mixed extract of 1 mg/ml), and the inhibition of mast cell degranulation was 90.1% or 70.6%, respectively (see Table 4).

From the above test results, it can be seen that, even when the Houttuynia cordata extract or the Rubus coreanus extract is used alone, the mast cell degranulation induced by compound 48/80 can be inhibited, but when the mixed extract of Houttuynia cordata and Rubus coreanus is used, the mast cell degranulation can be effectively inhibited.

TABLE 4
Effect of mixed extract of Houttuynia cordata and Rubus coreanus on
inhibition of degranulation of mouse mesenteric mast cells

| | | | Mast cells (%) | | | | |
|---|---|---|---|---|---|---|---|
| | | | | | | | Inhibition of |
| Treatment | | Compound | | Moderate | Severe | Degranulation | degranulation |
| (mg/ml) | | 48/80 | Normal | degranulation | degranulation | index | (%) |
| Saline | | − | 88.9 ± 1.6¹⁾ | 9.6 ± 2.6 | 1.5 ± 1.2 | 6.3 | — |
| | | + | 2.4 ± 0.8 | 9.9 ± 1.4 | 87.7 ± 4.8 | 92.7 | — |
| Houttuynia | 10 | + | 40.4 ± 3.3 | 35.1 ± 3.7 | 24.5 ± 2.9 | 42.0 | 54.6 |
| cordata | 1 | + | 8.8 ± 1.9 | 16.3 ± 3.3 | 73.9 ± 2.9 | 82.1 | 11.4 |
| extract | | | | | | | |
| Rubus | 10 | + | 39.4 ± 5.3 | 39.8 ± 4.0 | 22.8 ± 2.6 | 42.7 | 53.9 |
| coreanus | 1 | + | 9.3 ± 4.6 | 20.3 ± 4.2 | 70.4 ± 3.2 | 80.6 | 13.0 |
| extract | | | | | | | |
| Mixed | 10 | + | 84.9 ± 2.3 | 11.9 ± 2.0 | 3.2 ± 1.5 | 9.1 | 90.1 |
| extract of | 1 | + | 60.4 ± 5.3 | 24.8 ± 4.0 | 14.8 ± 3.6 | 27.2 | 70.6 |
| Houttuynia | | | | | | | |
| cordata, | | | | | | | |
| Rubus | | | | | | | |
| coreanus | | | | | | | |

+ treated,
− untreated.
¹⁾each value was expressed as mean ± standard error(n = 10)

### TEST EXAMPLE 5

### Examination of Antiallergic Activity of Mixed Extract of Houttuynia cordata and Rubus coreanus Using Cutaneous Reaction Rats Model

Compound 48/80 was administered into the dermis of healthy and mature Sprague-Dawley rats weighing 250-300 g to cause a cutaneous reaction. Then, whether the inventive mixed extract of Houttuynia cordata and Rubus coreanus inhibits the cutaneous reaction and skin vascular permeability was examined.

The test for causing the cutaneous reaction in rats was performed in the following manner by a method modified and supplemented from a known method (Byoung-Duek, Jeon et al., The Korean Journal of Biological Response Modifier, 2, 169, 1992). The back hair of male rats was removed, and then under ether anesthesia, 50 μl of each of 0.9% saline, 50 ml of 10 mg/ml of the Houttuynia cordata extract, the Rubus coreanus extract and the mixed extract of Houttuynia cordata and Rubus coreanus, was injected into the dermis of the back skin respectively. After 10 minutes, 50 μl of 5 μg/ml compound 48/80 was injected. As a control group, 50 μl of saline in place of the compound 48/80 solution was injected. At 20 minutes after injection with the compound 48/80, 400 μl of 0.5% Evan's blue solution was injected into the vein of the back of the penis of the rats. The determination of causing the cutaneous reaction, and the determination of a positive reaction were performed by incising the back skin 30 minutes after injection with the Evan's blue solution, and then observing whether blue spots on the dermis appear. And, the skin portion showing the blue spots was cut off and measured for its weight. Then, the skin portion was cut finely into small pieces of about 3-4 mm, put in 2 ml of formamide solution, and allowed to react in an oven at 80° C. for 3 hours to release the Evan's blue solution. The density of the released Evan's blue solution was measured with a spectrophotometer (spectra MAX plus, Molecular Devices, USA) at 620 nm, and the concentration of the Evan's blue was calculated by the Evans blue standard curve. Also, based on the calculated Evan's blue concentration, the inhibition of vascular permeability (%) was calculated by the following equation:
Inhibition of vascular permeability (%)=[1-(Evan's blue concentration by administration with herbal extract and compound 48/80−Evan's blue concentration by administration with herbal extract)/(Evan's blue concentration by administration with only compound 48/80−Evan's blue concentration by administration with only saline)]×100

In the test results, the control group administered with only saline did not showed blue spots on the skin, but the group administered with only compound 48/80 showed blue spots, indicating that a cutaneous reaction was caused. Also, the groups administered only with each of the Houttuynia cordata extract, the Rubus coreanus extract and the mixed extract of Houttuynia cordata and Rubus coreanus did not show blue spots. Thus, it can be found that saline and each of the extracts do not cause the skin reaction on the skin, and compound 48/80 causes the cutaneous reaction on the skin. Meanwhile, the group administered with each of the Houttuynia cordata extract, the Rubus coreanus extract and the mixed extract of Houttuynia cordata and Rubus coreanus and then with compound 48/80 showed a reduction in blue spots as compared to the group administered with only compound 48/80 of the same concentration.

The skin vascular permeability on the cutaneous reaction-caused site was quantitatively analyzed, and as a result, the case of administration with only compound 48/80 showed an Evan's blue concentration of 36.7±2.7 μg/g. The group administered with the Houttuynia cordata extract and then with compound 48/80 showed an Evan's blue concentration of 25.8±1.7 μg/g and an inhibition of 37% against the vascular permeability induced by compound 48/80. In the group administered with the Rubus coreanus extract and then with compound 48/80, the Evan's blue concentration was 23.3±1.5 μg/g, the vascular permeability induced by compound 48/80 was 45% inhibited. In the group administered with the mixed extract of Houttuynia cordata and Rubus coreanus and then with compound 48/80, the Evan's blue concentration was 8.7±0.8 μg/g, and the vascular permeability caused by compound 48/80 was 93% inhibited (see Table 5).

From the above test results, it can be seen that even when the Houttuynia cordata extract or the Rubus coreanus extract is used alone, the effect of inhibiting the cutaneous reaction and the vascular permeability increase induced by compound 48/80 can be obtained. Also, it can be seen that, when the mixed extract of Houttuynia cordata and Rubus coreanus is used, the cutaneous reaction and increase of vascular permeability can be more effectively inhibited.

TABLE 5
Effects of mixed extract of Houttuynia cordata and Rubus coreanus on
inhibition of cutaneous reaction and vascular permeability

| | | Evan's blue | Inhibition of |
|---|---|---|---|
| | Compound | concentration | vascular |
| Treatment | 48/80 | (μg/g) | permeability (%) |
| Saline | − | 6.4 ± 0.8¹⁾ | |
| Saline | + | 36.7 ± 2.7 | |
| Houttuynia cordata | − | 6.8 ± 0.7 | |
| extract | + | 25.8 ± 1.7 | 37 |
| Rubus coreanus | − | 6.6 ± 0.9 | |
| extract | + | 23.3 ± 1.5 | 45 |
| Mixed extract of | − | 6.7 ± 0.8 | |
| Houttuynia cordata | + | 8.7 ± 0.8 | 93 |
| and Rubus coreanus | | | |

+ treated,
− untreated
¹⁾each value was expressed as mean ± standard error (n = 10).

### EXAMPLE 4

### Preparation of Mixed Extract of Houttuynia cordata, Rubus coreanus, Cornus officinalis Sieb. et Zucc, Crataegi fructus and Mori folium

In addition to the Houttuynia cordata extract and the Rubus coreanus extract, Cornus officinalis Sieb. et Zucc, Crataegi fructus and Mori folium confirmed to have an inhibitory effect against degranulation of mast cells in Test Example 1 were further added to prepare a mixed extract. Houttuynia cordata, Rubus coreanus, Cornus officinalis Sieb. et Zucc, Crataegi fructus and Mori folium in Example 1 were mixed with each other at a weight ratio of 16:16:1:1:1, from which a mixed extract was prepared in the following manner. Houttuynia cordata, Rubus coreanus, Cornus officinalis Sieb. et Zucc, Crataegi fructus and Mori folium selected in Test Example 1 were washed clean and cut into a constant size of less than 1×1×1 cm. Then, 350 g of a herbal mixture consisting of 160 g of the Houttuynia cordata, 160 g of the Rubus coreanus, 10 g of the Cornus officinalis Sieb. et Zucc, 10 g of the Crataegi fructus and 10 g of the Mori folium was put into a 3 liters of purified water and extracted under pressure at 100° C. for 4 hours.

### TEST EXAMPLE 6

### Examination of Effect of Mixed Extract of Houttuynia cordata, Rubus coreanus, Cornus officinalis Sieb. et Zucc, Crataegi fructus and Mori folium on Inhibition of Histamine Release from Mast Cells

Whether the mixed extract of Houttuynia cordata, Rubus coreanus, Cornus officinalis Sieb. et Zucc, Crataegi fructus and Mori folium prepared in Example 4 has the effect of inhibiting histamine release from mast cells was examined. The examination of the histamine release-inhibitory effect was performed in the same manner as in Test Example 3. In this case, the final treatment concentrations of the mixed extract treated on mast cells were 0.01, 0.025, 0.05, 0.1, 1.0 and 10.0 mg/ml, respectively.

In the test results, in the case of the mast cell suspension added with saline, the histamine release was 3.1%. In the case of the mast cells treated with only the mixed extract of Houttuynia cordata, Rubus coreanus, Cornus officinalis Sieb. et Zucc, Crataegi fructus and Mori folium, the histamine release was similar to that in the mast cells treated with only saline. On the other hand, the mast cells treated with only compound 48/80 showed a very high histamine release of 56.7%. Also, in the case of the mast cells treated with the mixed extract followed by compound 48/80, the histamine release from the mast cells induced by compound 48/80 was inhibited (see Table 6).

TABLE 6
Effect of mixed extract of Houttuynia cordata, Rubus coreanus, Cornus
officinalis Sieb. et Zucc, Crataegi fructus and Mori folium on
inhibition of histamine release

| Final treatment | Final treatment | | Inhibition of |
|---|---|---|---|
| concentration of | concentration of | Histamine | histamine |
| mixed extract | compound 48/80 | release (%) | release (%) |
| 0 | 0 | 3.1 ± 0.9¹⁾ | |
| 0 | 0.5 | 56.7 ± 2.5 | |
| 0.01 | 0.5 | 37.2 ± 2.8 | 37 |
| 0.025 | 0.5 | 20.2 ± 1.9 | 68 |
| 0.05 | 0.5 | 19.7 ± 1.5 | 69 |
| 0.1 | 0.5 | 17.4 ± 0.5 | 74 |
| 1 | 0.5 | 15.0 ± 0.9 | 79 |
| 10 | 0.5 | 11.5 ± 0.3 | 86 |

¹⁾each value was expressed as mean ± standard error (n = 10).

### EXAMPLE 5

### Preparation of Capsules Comprising Mixed Extracts of Herbal Materials According to the Present Invention

Capsules comprising a mixed extract of Houttuynia cordata and Rubus coreanus as an active ingredient were prepared. Also, capsules comprising a mixed extract of Houttuynia cordata, Rubus coreanus, Cornus officinalis Sieb. et Zucc, Crataegi fructus and Mori folium as an active ingredient were prepared.

The capsules comprising the mixed extract of Houttuynia cordata and Rubus coreanus were prepared in the following manner. 100 mg of the powdered Houttuynia cordata extract and 100 mg of the powdered Rubus coreanus extract, prepared in Example 1, and 100 mg of lactose, were precisely weighed, and homogeneously mixed in a mixer. Then, the mixture was filled into hard gelatin capsules (Su-Heung Capsule Co., Ltd., Korea) with an automatic capsule filling machine at 300 mg for each capsule.

The capsules comprising the mixed extract of Houltuynia cordata, Rubus coreanus, Cornus officinalis Sieb. et Zucc, Crataegi fructus and Mori folium were prepared in the following manner. 80 mg of the powdered Houttuynia cordata, 80 mg of the powdered Rubus coreanus extract, 10 mg of the powdered Cornus officinalis Sieb. et Zucc extract, 10 mg of the powdered Crataegi fructus extract and 10 mg of the powdered Mori folium extract, prepared in Example 1, and 110 mg of lactose, were precisely weighed, and homogeneously mixed in a mixer. Then, the mixture was filled into hard gelatin capsules (Su-Heung Capsule Co., Ltd., Korea) with an automatic capsule filling machine at 300 mg for each capsule.

### TEST EXAMPLE 7

### Verification of Anti-allergic Effect of the Inventive Herbal Extract Formulations by Clinical Test

The verification of the anti-allergic effect of the herbal extract formulations prepared in Example 5 was performed on allergic disease patients.

7-1) Classify into Clinical Test Groups

Clinical tests on twenty 10-70 years old men and women patients suffering from atopic dermatitis were performed. First, the 20 patients were classified into four groups consisting of a group administered with the inventive formulation 1 (containing the mixed extract of Houttuynia cordata and Rubus coreanus, see Table 7), a group administered with the inventive formulation 2 (containing the mixed extract of Houttuynia cordata, Rubus coreanus, Cornus officinalis Sieb. et Zucc, Crataegi fructus and Mori folium, see Table 7), a group administered with the conventional therapeutic agent (see Table 8), and a group administered with the inventive formulation 1 (containing the mixed extract of Houttuynia cordata and Rubus coreanus along with the conventional therapeutic agent, see Table 9), each group consisting of five patients.

As the conventional therapeutic agents, the following steroid agents were used: steroid agents such as advantan (Schering Ltd.) lacticare (Stifel Laboratories) esperson gel (Handok Pharmaceuticals Co. Ltd.) oradexion (Choong Wae Pharma Corporation), dexcosil (Yungjin Paramaceutical Co. Ltd.); antihistamin agents such as plokon (Yungjin Parama. Co. Ltd.), primalan (Bukwang Pharmaceutical Co. Ltd.), allegra (Handok Pharmaceuticals Co. Ltd.), zaditen (Novartis Pharmaceuticals), remicut (Kolon Pharmaceuticals, Inc.); and epogam (Handok Pharmaceuticals Co. Ltd.) that is evening primrose oil used as an auxiliary therapeutic agent against atopic dermatitis.

TABLE 7
Group administered with the inventive formulation

| | | Dose/ | Administration |
|---|---|---|---|
| | Age/sex | administration mode | period |
| Case 1 | 8 years old/man | 2 capsules/day | More than 2 weeks |
| | | (600 mg/day), oral | |
| Case 2 | 12 years old/woman | 2 capsules/day | More than 2 weeks |
| | | (600 mg/day), oral | |
| Case 3 | 22 years old/man | 4 capsules/day | More than 2 weeks |
| | | (1.2 g/day), oral | |
| Case 4 | 27 years old/woman | 4 capsules/day | More than 2 weeks |
| | | (1.2 g/day), oral | |
| Case 5 | 7 years old/man | 2 capsules/day | More than 2 weeks |
| | | (600 mg/day), oral | |

TABLE 8
Group administered with the conventional therapeutic agents

| | | | Administration |
|---|---|---|---|
| | Age/sex | Kind of therapeutic agent and dose | mode |
| Case 1 | 8 years | 2.5 mg oradexion and 1.5 mg plokon, one | Muscular injection |
| | old/man | times/week, 2 times/week (severe condition) | |
| | | Lacticare ointment, 2-3 times/week | Transdermal |
| | | Primalan syrup, 2 times/day, 12.5 ml for each time | Oral |
| Case 2 | 10 years | 2.5 mg oradexion and 1.5 mg plokon, one | Muscular injection |
| | old/woman | time/week, two times/week (severe condition) | |
| | | Advantan ointment, 2 times/day, severe sites | Transdermal |
| | | Lacticare ointment, 2-3 times/day, non-severe | Transdermal |
| | | sites | |
| | | Epogam, two times/day, 320 mg for each time | Oral |
| | | Allegra, two times/day, 60 mg for each time | Oral |
| Case 3 | 35 years | 5 mg oradexion and 3 mg plokon, one time/week | Muscular injection |
| | old/man | three times/week (severe condition) | |
| | | Advantan ointment, two times/day | Transdermal |
| | | Lacticare ointment, 2-3 times/day | Transdermal |
| | | Epogam, two times/day, 320 mg for each time | Oral |
| | | Allegra, two times/day, 180 mg for each time | Oral |
| Case 4 | 22 years | 5 mg oradexion and 3 mg plokon, one time/week, | Muscular injection |
| | old/woman | two times/week (severe condition) | |
| | | Advantan ointment, two times/ day (severe sites) | Transdermal |
| | | Lacticare ointment, 2-3 times/day (entire) | Transdermal |
| | | Epogam, two times/day, 320 mg for each time | Oral |
| | | Allegra, two times/day, 180 mg for each time | Oral |
| Case 5 | 24 years | 5 mg oradexion and 3 mg plokon, one time/week, | Muscualr injection |
| | old/woman | two times/week (severe condition) | |
| | | Esperson gel, two times/day (severe sites) | Transdermal |
| | | Epogam, two times/day, 320 mg for each time | Oral |
| | | Allegra, two times/day, 180 mg/each time | Oral |
| | | Zaditen, two times/day, 2 mg for each time | Oral |

TABLE 9
Group administered with the inventive formulation along with the
conventional therapeutic agents

| | | Dose/administration mode | | |
|---|---|---|---|---|
| | | Inventive | | Adminstration |
| | Age/sex | formulation | Conventional therapeutic agent | period |
| Case 1 | 31 months | 2 capsules/day | Advantan ointment, one time/day (severe | More than 2 |
| | old/woman | (600 mg/day), oral | sites) | weeks |
| | | | Dexcosil ointment, one time/day (around | |
| | | | face, eye) | |
| | | | Lacticare ointment, 1-2 times/day (non- | |
| | | | severe sites) | |
| | | | Epogam, two times/day, 160 mg/each time | |
| | | | Primalan syrup, two times/day, 7.5 ml for | |
| | | | each time | |
| Case 2 | 37 years | 4 capsules/day | 3 mg Plokon injection, one time/week | More than 2 |
| | old/man | 1.2 g/day, oral | (severe sites) | weeks |
| | | | two times/week (severe condition) | |
| | | | Adantan ointment, two times/day (severe | |
| | | | sites) | |
| | | | Lacticare ointment, 2-3 times/day (entire) | |
| | | | Allegra, one time/day, 180 mg for each | |
| | | | time | |
| | | | Remicut, two times/day, 2 mg for each | |
| | | | time | |
| Case 3 | 9 years | 2 capsules/day | 2.5 mg oradexion and 1.5 mg plokon | More than 2 |
| | old/woman | (600 mg/day), oral | injection, one time/week | weeks |
| | | | Lacticare ointment, 2-3 times/day (entire) | |
| | | | Primalan syrup, two times/day, 12.5 ml for | |
| | | | each time | |
| Case 4 | 25 years | 4 capsules/day | 5 mg oradexion and 3 mg plokon, one | More than 2 |
| | old/woman | (1.2 g/day), oral | time/week, two times/week (severe | weeks |
| | | | condition) | |
| | | | Esperson gel, two times/day (sites having | |
| | | | lesions) | |
| Case 5 | 22 years | 4 capsules/day | 5 mg oradexion and 3 mg plokon, one | More than 2 |
| | old/man | (1.2 g/day), oral | time/week | weeks |
| | | | Esperson gel, two times/day (sites having | |
| | | | lesions) | |

7-2) Measurement of IgE and Histamine Levels in Serum of Allergic Disease Patients

The blood of patients of each test group in Test Example 7-1) was collected before and after treatment, and the IgE and histamine levels in the serum were measured. The measurement of the IgE level was performed by ECLIA (electrochemiluminescence immunoassay). An analytic kit containing a complex of a biotinylated monoclonal IgE-specific antibody and a monoclonal IgE-specific antibody labeled with a ruthenium was used in the measurement. The measurement of the histamine level in the serum of the patients was performed in the same manner as in Test Example 3.

In the test results, all the test groups after treatment showed reductions in the serum IgE and histamine levels as compared to before treatment (see Tables 10 and 11). Also, there was no great difference between the group administered with the inventive formulation, the group administered with the conventional therapeutic agents, and the group administered with the inventive formulation along with the conventional therapeutic agents.

TABLE 10
IgE levels in serum of allergic disease patients (IU/ml)

| | | | | | Group administered with | |
|---|---|---|---|---|---|---|
| | Group administered | | Group administered with | | the inventive formulation | |
| | with the inventive | | the conventional | | along with the conventional | |
| | formulation | | therapeutic agents | | therapeutic agent | |
| | Before | After | Before | After | Before | After |
| Case/group | treatment | treatment | treatment | treatment | treatment | treatment |
| 1 | 192.6 | 150.2 | 26.5 | 23.1 | 41.1 | 36.1 |
| 2 | 57.4 | 48.2 | 460.7 | 409.9 | 500 | 487 |
| 3 | 75.9 | 68.4 | 47.5 | 41.9 | 135.1 | 121.8 |
| 4 | 42.5 | 40.3 | 32.7 | 29.7 | 28.7 | 25.9 |
| 5 | 339.6 | 305.4 | 55.7 | 55.0 | 19.3 | 16.7 |

TABLE 11
Histamine levels in serum of allergic disease patients (nM)

| | | | | | Group administered with | |
|---|---|---|---|---|---|---|
| | Group administered | | Group administered with | | the inventive formulation | |
| | with the inventive | | the conventional | | along with the conventional | |
| | formulation | | therapeutic agents | | thereapeutic agent | |
| | Before | After | Before | After | Before | After |
| Case/group | treatment | treatment | treatment | treatment | treatment | treatment |
| 1 | 135 ± 5 | 125 ± 5 | 140 ± 10 | 135 ± 5 | 145 ± 15 | 145 ± 5 |
| 2 | 140 ± 5 | 135 ± 10 | 165 ± 5 | 140 ± 10 | 140 ± 10 | 140 ± 10 |
| 3 | 150 ± 15 | 145 ± 10 | 135 ± 10 | 130 ± 10 | 135 ± 5 | 130 ± 10 |
| 4 | 135 ± 10 | 130 ± 10 | 140 ± 15 | 140 ± 5 | 140 ± 5 | 130 ± 10 |
| 5 | 155 ± 5 | 145 ± 10 | 125 ± 10 | 120 ± 10 | 130 ± 10 | 125 ± 10 |

7-3) Clinical Observation of Allergic Disease Patients

Lesions of patients of each test group in Test Example 7-1) were clinically observed. Atopic dermatitis is characterized by various lesions, including the development of erythematous papules and vesicles with severe pruritus, the development of exudative lesions upon scratching, the development of excoriation, erythematous or scaled papules and plaques, and the development of lichenification resulting from repeated scratching and rubbing. The improvement of atopic dermatits was determined by observing an increase or decrease in such characteristics and symptoms.

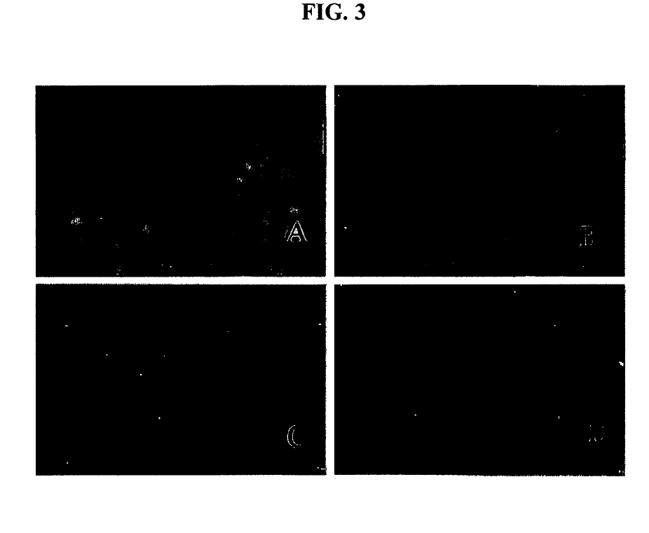

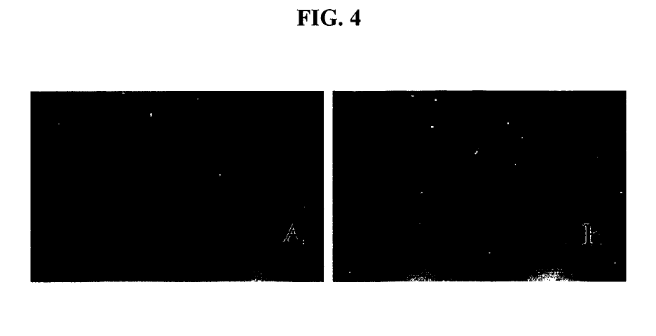

In the test results, the atopic dermatitis patients administered with the inventive formulation 1 or 2 showed the improvement of lichenoid lesions involving fissures on the palm (see A and B of FIG. 3), and showed the improvement of round erythematous macules of popliteal fossae (see FIG. C and D of FIG. 3). Also, the test group administered with the conventional therapeutic agents showed the improvement of round erythematous plaques of antecubital fossae and the improvement of eczematous lesions accompanied by scales on the face (see A and B of FIG. 4). In addition, the test group administered with the conventional therapeutic agents along with the inventive formulation showed the improvement of eczematous lesions accompanied by scales on the face.

Accordingly, it can be found that the composition comprising the inventive herbal extracts has substantially the same effect as the conventional steroid agents on the treatment of allergic diseases.

### EXAMPLE 6

### Preparation of Beverage Composition Comprising Herbal Extracts According to the Present Invention

A beverage composition was prepared by mixing 20% of the Houttuynia cordata extract, 20% of the Rubus coreanus extract, 0.15% of vitamin A, 0.2% of vitamin D, 0.15% of vitamin B2, 2,0% of vitamin C, 3.0% of taurin, 2.5% of polydextrose, and the remainder of purified water.

The entire disclosure of Korea Patent Application No. 2004-0007404, filed on Feb. 5, 2004 including its specification, claims, drawings and summary are incorporated herein by reference in its entirety.

### INDUSTRIAL APPLICABILITY

As can be seen from the foregoing, the inventive mixed extract of Houttuynia cordata and Rubus coreanus and the composition comprising the same have the effect of preventing and treating allergic diseases by inhibiting the degranulation and histamine release of mast cells. Also, they show no cytotoxicity and thus can be safely used in vivo.

1. Herbal extract having inhibitory activities against the degranulation and histamine release of mast cells, which is obtained by extracting Houttuynia cordata and Rubus coreanus with water or organic solvent.

2. The herbal extract of claim 1, wherein the Houttuynia cordata and the Rubus coreanus are mixed at a weight ratio of 1:4 to 4:1.

3. The herbal extract of claim 1, which further comprises at least one selected from the group consisting of Cornus officinalis Sieb. et Zucc, Crataegi fructus and Mori folium.

4. The herbal extract of claim 1 for use as medicament.

5. A use of the herbal extract of claim 1 for the preparation of therapeutic agents against allergic diseases.

6. A use of the herbal extract of claim 1 for the preparation of inhibitory agents against the degranulation and histamine release of mast cells.

7. A pharmaceutical composition for the prevention or treatment of allergic diseases comprising the herbal extract of claim 1 as an active ingredient.

8. A food composition for the prevention or improvement of allergic diseases comprising the herbal extract of claim 1 as an active ingredient.

9. The composition of claim 7, which further comprises at least one selected from the group consisting of Cornus officinalis Sieb. et Zucc extract, Crataegi fructus extract and Mori folium extract.

10. The composition of claim 7, wherein the allergic diseases are selected from the group consisting of allergic asthma, allergic rhinitis, allergic otitis, anaphylactic shock and allergic skin disorders.

11. The composition of claim 10, wherein the allergic skin disorders are selected from the group consisting of atopic dermatitis, psoriasis, contact allergic dermatitis and urticaria.

12. A method for preventing or treating allergic diseases, which comprises the step of administering an effective amount of the herbal extract of claim 1 to a subject in need thereof.

13. A method for inhibiting the degranulation and histamine release of mast cells, which comprises the step of administering an effective amount of the herbal extract of claim 1 to a subject in need thereof.

14. The herbal extract of claim 2, which further comprises at least one selected from the group consisting of Cornus officinalis Sieb. et Zucc, Crataegi fructus and Mori folium.

15. The composition of claim 8, which further comprises at least one selected from the group consisting of Cornus officinalis Sieb. et Zucc extract, Crataegi fructus extract and Mori folium extract.

16. The composition of claim 8, wherein the allergic diseases are selected from the group consisting of allergic asthma, allergic rhinitis, allergic otitis, anaphylactic shock and allergic skin disorders.
